(19) United States
(12) Patent Application Publication
(10) Pub. No.: US 2026/0065106 A1
(43) Pub. Date: Mar. 5, 2026

(54) SITUATIONAL AWARENESS UNCERTAINTY PROPAGATION FOR MEDICAL DECISION MAKING

(71) Applicant: NEC Laboratories America, Inc., Princeton, NJ (US)

(72) Inventors: Xujiang Zhao, Hillsborough, NJ (US); Yiyou Sun, Madison, WI (US); Yanchi Liu, Princeton, NJ (US); Wei Cheng, Princeton Junction, NJ (US); Haifeng Chen, West Windsor, NJ (US); Qiwei Zhao, Kendall Park (IS)

(21) Appl. No.: 19/317,317

(22) Filed: Sep. 3, 2025

Related U.S. Application Data
(60) Provisional application No. 63/690,821, filed on Sep. 5, 2024.

(51) Int. Cl. G06N 7/01; G16H 50/20

(52) U.S. Cl. CPC G06N 7/01; G16H 50/20

(57) ABSTRACT

Methods and systems include estimating situational weights for an agent based on a distance measure for steps taken by the agent. The situational weights are combined with uncertainties from the agent for the steps to determine a total uncertainty for an action indicated by the agent. The action indicated by the agent is performed responsive to the total uncertainty.

### RELATED APPLICATION INFORMATION

This application claims priority to U.S. Patent Application No. 63/690,821, filed on Sep. 5, 2024, incorporated herein by reference in its entirety.

### BACKGROUND

### Technical Field

The present invention relates to large language model (LLM) agents and, more particularly, to quantifying uncertainty for agents.

### Description of the Related Art

LLM-based agent systems can perform actions according to a user's directions, but their outputs are not always correct, which can have negative consequences in high-stakes scenarios. Determining the reliability of an LLM agent's outputs is challenging.

### SUMMARY

A method includes estimating situational weights for an agent based on a distance measure for steps taken by the agent. The situational weights are combined with uncertainties from the agent for the steps to determine a total uncertainty for an action indicated by the agent. The action indicated by the agent is performed responsive to the total uncertainty.

A system includes a hardware processor and a memory that stores a computer program. When executed by the hardware processor, the computer program causes the hardware processor to estimate situational weights for an agent based on a distance measure for a plurality of steps taken by the agent, to combine the situational weights with uncertainties from the agent for the plurality of steps to determine a total uncertainty for an action indicated by the agent, and to perform the action indicated by the agent responsive to the total uncertainty.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram showing the operation of a large language model (LLM) agent using uncertainty estimation to judge the reliability of the agent's outputs, in accordance with an embodiment of the present invention;

FIG. 2 is a block diagram showing how uncertainty is estimated using situational weight estimation based on information from the LLM agent, in accordance with an embodiment of the present invention;

FIG. 3 is a block diagram illustrating the use of a continuous hidden Markov model to combine distance information from the LLM agent, in accordance with an embodiment of the present invention;

FIG. 4 is pseudo-code showing uncertainty estimation using situational estimation, in accordance with an embodiment of the present invention;

FIG. 5 is a block diagram of a healthcare facility that uses an agent with situational awareness uncertainty propagation, in accordance with an embodiment of the present invention;

FIG. 6 is a block diagram of a computing device that can implement an LLM agent with uncertainty estimation, in accordance with an embodiment of the present invention;

FIG. 7 is a diagram of an exemplary neural network architecture that can be used to implement part of the LLM agent, in accordance with an embodiment of the present invention; and

FIG. 8 is a diagram of an exemplary deep neural network architecture that can be sued to implement part of the LLM agent, in accordance with an embodiment of the present invention.

### DETAILED DESCRIPTION OF PREFERRED EMBODIMENTS

The uncertainty of a large language model (LLM) agent's outputs can be calculated to determine the reliability of the agent's actions. The uncertainty of an agent's outputs may accumulate gradually, such that the uncertainty at any time is greatly influenced by interactions with the environment. Situation awareness uncertainty may be propagated through time, where the real uncertainty of the LLM-based agent is estimated in a multi-step interaction with the environment. Surrogates may further be used to estimate the agent's directly un-computed situation, including position-based, distance-based, and hybrid versions.

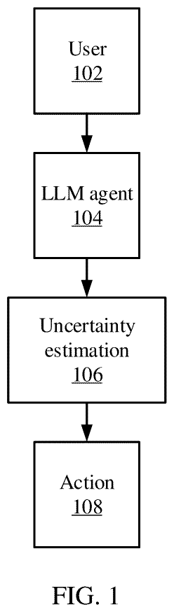

Referring now to FIG. 1, an LLM-based system that uses situation awareness uncertainty propagation is shown. A user 102 provides instructions to an LLM agent 104, for example in the form of a natural language prompt. The LLM agent 104 makes a determination about how to execute the user's instructions, for example drawing on a knowledge base or trained information, and generates an output. Uncertainty estimation 106 is performed on the output to determine its reliability.

Based on the uncertainty estimation 106, an action 108 is performed that accounts for the reliability of the output. For example, for a highly reliable output, the action 108 may be executed as indicated by the LLM agent 104. For a highly unreliable output, the action 108 may include a report back to the user 102 indicating that their instructions cannot be confidently fulfilled. For an output with a reliability between these extremes, the action 108 may include the collection of additional information or the performance of mitigation acts which reduce the risk of an incorrect output.

For example, the estimated uncertainty may be compared to a first threshold value and a second threshold value. If the uncertainty exceeds the first threshold value, then the action 108 may not be performed. If the uncertainty falls below the second threshold value

The LLM agent 104 may predict an answer label y∈ [symbol] based on an input x∈ [symbol] . Given a parameter space Θ, an LLM L_{θ} may be initialized with the parameters θ∈Θ. The LLM may be implemented as a sequential model that produces structured outputs. Both its inputs and outputs may be viewed as sequences of tokens:

$${L}_{θ} ( x ) = {L}_{θ} ( {t}_{{x}_{1}} , … , {t}_{{x}_{n}} )$$

Each tₓ_{ᵢ} is a token of the input. The output sequence is denoted as

$${L}_{θ} ( x ) = [ {t}_{{y}_{1}} , … , {t}_{{y}_{n}} ]$$

The LLM agent 104 may be abstracted as a pipeline of the agent L_{θ}, the problem [symbol] , the environment E, an observation O, an action A, and a thought process T, where O satisfies O=E(A). For an n^{th} step, the agent determines the current Tₙ and Aₙ based on the obtained O₁, . . . , Oₙ₋₁ and the preceding A₁, . . . , Aₙ₋₁, T₁, . . . , Tₙ₋₁. At each step, the LLM agent will first consider the prompt and then take an action, where the observation O is the output of the action. For example, if the action is to search for a term in a knowledge base, the observation may be the output of the search. The agent can either continue interacting with the environment to acquire new O or can confidently produce a final output based on the current results. For step n, the sequence of all A, T, and O from step 1 to step (n−1) is defined as Zₙ₋₁:

$${T}_{N} = {L}_{θ} ( Q , {Z}_{n - 1} )$$

$${Z}_{n - 1} = \{ ( {A}_{1} , {T}_{1} , {O}_{1} ) , ( {A}_{2} , {T}_{2} , {O}_{2} ) , … , ( {A}_{n - 1} , {T}_{n - 1} , {O}_{n - 1} ) \}$$

The uncertainty estimation 106 assigns higher uncertainty values when the LLM agent's predictions are likely to be incorrect and assigns lower uncertainty values otherwise. Predictive entropy may be used for uncertainty estimation, measuring the information about the output given the input. The predictive entropy may be determined as:

$$PE ( x ) = H ( {L}_{θ} \vert x ) = - \int p ( {L}_{θ} ( x ) \vert x ) ln ( p ( {L}_{θ} ( x ) \vert x ) ) {dL}_{θ} ( x )$$

The predictive entropy may thus be calculated with a distribution of the tokens. Normalized entropy is an alternative to predictive entropy, using the likelihood or the normalized likelihood of the output tokens as the measure of the overall uncertainty:

$$NE ( {L}_{θ} ( x ) ) = \frac{1}{N} {\prod }_{i \leq N} p ( {t}_{i} \vert {t}_{1} , … , {t}_{i - 1} ; θ ) = \frac{1}{N} {\prod }_{i \leq N} log p ( {t}_{i} \vert {t}_{1} , … , {t}_{i - 1} ; θ )$$

Further alternatives include leveraging the strong instruction-following capabilities of LLMs, asking the LLM agent 104 about its own uncertainty after the final step to estimate the feasibility of the preceding process.

The system's hidden states may be observed based on observable states using a hidden Markov model. The model assumes that system transitions between these hidden states according to certain regularities. N may denote the number of hidden states and M may denote the number of observable states. The target system's hidden states are characterized as Sₕₘₘ={Sₕₘₘ_{₁}, . . . , Sₕₘₘ_{_{N}}} and a set of observable states Oₕₘₘ={Oₕₘₘ_{₁}, . . . , Oₕₘₘ_{_{N}}}.

The state transition probability matrix A=[aᵢⱼ] may be defined where aᵢⱼ=P(Sₕₘₘ_{ᵢ}|Sₕₘₘ_{ⱼ}) represents the probability of transitioning from state Sₕₘₘ, to state Sₕₘₘ_{ⱼ}. The observation probability matrix B=[bⱼₖ] is defined where bⱼₖ=P(Oₕₘₘ_{ₖ}|Sₕₘₘ_{ⱼ}) represents the probability of observing Oₕₘₘ_{ₖ} given the system is in state Sₕₘₘ_{ⱼ}. The initial state distribution π=[πᵢ] is defined where πᵢ=P(Sᵢ) represents the probability of the system starting in state Sₕₘₘ_{ᵢ}.

A hidden Markov model is fully specified by the triplet (π,A,B). A continuous hidden Markov model is a specialized version of the hidden Markov model. While observations are discrete in the traditional hidden Markov model, they are continuous in the continuous hidden Markov model. For each state, the observation probability is modeled by a continuous probability density function. Gaussian mixture models may be used to model the observation probability B for a continuous hidden Markov model.

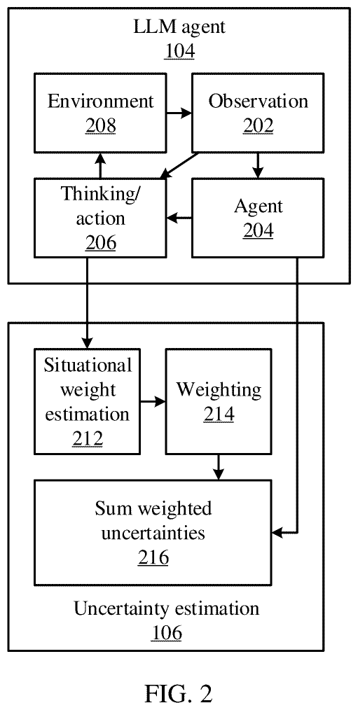

Referring now to FIG. 2, additional detail is provided on the LLM agent 104 and the uncertainty estimation 106 to illustrate a situational awareness uncertainty propagation pipeline that accurately estimates the agent's uncertainty by comprehensively considering the uncertainty at each step and the corresponding situational weights.

The LLM agent 104 may be implemented as a multi-step agent 204, which includes the behaviors of thinking, action, and observation of the environment 208. A distance Dₐ is measured between an input question and the combination of thinking, action, and observation. A distance D₀ denotes the distance between the observation 202 and the thinking/action 206. The agent 204 generates an answer responsive to an input question.

The uncertainty estimation 106 takes the D₀ and Dₐ measurements to determine an uncertainty for the agent's answer. As shown in greater detail below, situational weight estimation 212 uses a hidden Markov model to estimate the situational weights based on distances Dₐ and D₀. The situational weights are combined with the uncertainties from the agent 204 by pointwise multiplication 214 to determine weighted uncertainties, which are then summed 216 to arrive at the agent's uncertainty.

For each step i the agent 204 provides the thinking/action 206 with corresponding uncertainty Uᵢ based on a previous state Zᵢ₋ₜ and question [symbol] . Considering only the uncertainty of the last step as the overall uncertainty U_{agent} would not be comprehensive. Instead, the uncertainties of all steps may be considered, for example using an arithmetic mean. If the agent 204 gives the final answer after N steps, then this mean may be formulated as

$${U}_{agent} = \frac{{\sum }_{i \leq N} {U}_{i}}{N}$$

For robustness against outliers, accurate reflection of central tendency, and consistency in proportional changes, the geometric mean or root mean square may be a better choice compared to the arithmetic mean:

$${U}_{agent} = {( {\prod }_{i = 1}^{N} {U}_{i} )}^{\frac{1}{N}}$$

$${U}_{agent} = \sqrt{\frac{1}{N} {\sum }_{i = 1}^{N} {U}_{i}^{2}}$$

The contribution of uncertainty at different steps to the overall agent uncertainty is not uniform. During the process of obtaining the final answer, the agent 204 generates uncertainties. The contribution of the current step's uncertainty to the overall uncertainty, due to the agent's situation, is referred to herein as a situational weight. Situational weights are determined by such factors as deviations from the appropriate logical path and the quality of interactions between the agent 204 and the environment 208, which influence the correctness of the final answer. The situational weights are variable during the agent's problem-solving process and its interaction with the environment. Assuming that the uncertainty at step i is Uᵢ and the corresponding situational weight is Wᵢ, then the agent's uncertainty may be formulated as:

$${U}_{agent} = \sqrt{\frac{1}{N} {\sum }_{i = 1}^{N} ( {γ ( {W}_{i} {U}_{i} )}^{2} + ( 1 - γ ) log ( {W}_{i} {U}_{i} + 1 ) )}$$

The weight factor γ is used for the linear and logical items. This formulation comprehensively considers all steps of the agent based on propagation. By introducing situational weights for the uncertainty of different steps, the impact of specific steps may be adjusted according to the specifics of that uncertainty. This pipeline better illustrates how uncertainty is accumulated and generated throughout the multi-step problem-solving process.

Because the optimal problem-solving path is unknown, measurable quantities that are related to the situational weights may be used as surrogates. Steps which are closer to the final answer are therefore assigned higher weights. The position of a step i in the problem-solving process, out of a total of N steps, can be used as a surrogate for the situational weight. Considering potential boundary effects, the weights should grow quickly at first and then slow down. The uncertainty of all the steps closer to the final answer will therefore have a relatively large impact on the final uncertainty. For example:

$${W}_{i} = \frac{ln ( 1 + α \frac{i}{N} )}{{\sum }_{j \leq N} ln ( 1 + α \frac{j}{N} )}$$

where α is a tradeoff parameter.

Apart from simply considering the position of the step corresponding to uncertainty, the degree of deviation between the real problem solving process and an optimal trajectory may be used as situational weights. A larger deviation should correspond to a greater uncertainty in the overall uncertainty propagation. Here Z refers to the trajectory of the agent as described above. The distance surrogate may then be defined as:

$${d}_{i} = Dis ( {Z}_{i} , {Z}_{i - perfect} )$$

$${W}_{i} = \frac{{d}_{i}}{{\sum }_{j \leq N} {d}_{j}}$$

However, this formula suffers from Z_{i-perfect} being difficult to obtain directly. An extra surrogate is therefore used instead. The distance is primarily influenced by deviations in the problem-solving process and the quality of the interaction between the agent 204 and the environment 208, which can be used as surrogates for the agent's situation. The distance surrogate can then be expressed as Dis(Zᵢ,Q)+Dis(Aᵢ,Oᵢ). The inverse of the matching score can be used to implement the function Dis(⋅,⋅).

The surrogate methods described above are suitable for different scenarios, but they can be considered in a hybrid approach:

$${W}_{{PD}_{i}} = α {W}_{{P}_{i}} + ( 1 - α ) {W}_{{D}_{i}}$$

where α is a weight factor (distinct from the tradeoff parameter above) between the situational weights from different surrogate strategies, W_{P}_{ᵢ} is a position weight, and W_{D}_{ᵢ} is a distance weight.

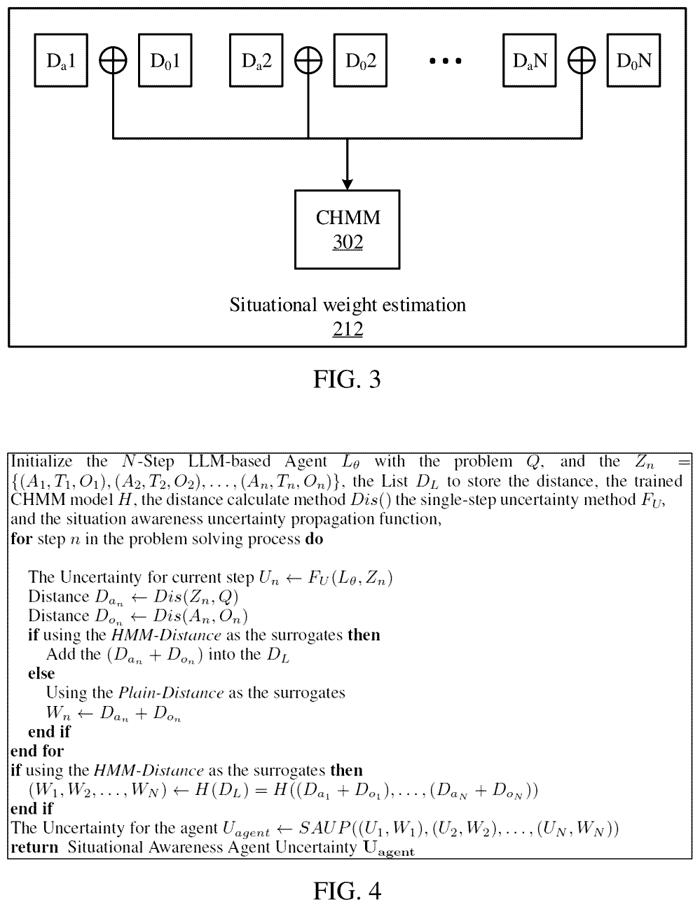

Referring now to FIG. 3, additional detail is shown for the situational weight estimation 212. The plain distance surrogate is feasible, but in some circumstances the calculated distances Dₐ and D₀ may remain small even when the agent's situation has deviated significantly from the correct trajectory to the final answer. This indicates that using only the plain distance as a surrogate for the agent 204 is not a comprehensive assessment. Situation weights may instead be used as hidden states for a hidden Markov model to provide a more thorough estimate.

A continuous hidden Markov model 302 is used to obtain a distance D_{HMM} that better serves as a surrogate for the situational weights of the agent 204. The continuous hidden Markov model may be defined with three discrete hidden states Sₕₘₘ, including the right trajectory to the answer, the relatively deviated trajectory, and the completely deviated trajectory, noted as conditions 0, 1, and 2 respectively. The observable states may be continuous quantities.

Here the plain distance Dₐ+D₀ is used and the transition probability matrix A, the observation probability matrix B, and the initial state distribution w are specified. Some examples are used to calculate Dₐ+D₀ and the corresponding hidden states are noted. The triplet (π,A,B) of the hidden Markov model is fully specified by the Baum-Welch algorithm. After training the continuous hidden Markov model 302, it can be used to transform the plain distance Dₐ+D₀ into D_{HMM}.

Referring now to FIG. 4, pseudo-code for situational awareness uncertainty propagation is shown. As described above, different surrogate formulations can be used. This pseudo-code uses distance as the surrogate. Initially, the uncertainty Un is computed for step n, along with the corresponding distances Dₐ_{ₙ} and D₀_{ₙ}. This procedure is repeated for N steps. Situational weights Wₙ are determined based on the distance surrogate choice, whether plain or generated by the hidden Markov model. The uncertainties U and weights W are aggregated to propagate situational awareness uncertainty U_{agent}.

The single-step uncertainty may use a modified normalized entropy. The normalized entropy can be applied to open-source LLMs, where complete logits of the output are accessible, as well as to LLMs that are accessible only via their API (application programming interface). It is additionally computationally efficient and demonstrates strong predictive performance for single-step uncertainty estimation.

For a step n and question [symbol] , the agent's thinking can be represented as Tₙ, with corresponding action Aₙ. The observation Oₙ is information gained from the environment through the action Aₙ. With an LLM L_{θ} and a trajectory of the previous steps Zₙ₋₁, the LLM will output the response of its thinking Tₙ and the action Aₙ together as:

$$( {T}_{n} , {A}_{n} ) = {L}_{θ} ( Q , {Z}_{n - 1} )$$

The step uncertainty

$${U}_{n} = {U}_{n}^{T} + {U}_{n}^{A}$$

is the combination of thinking uncertainty

$${U}_{n}^{T}$$

and action uncertainty

$${U}_{n}^{A} .$$

Predictive entropy with length normalization estimates the thinking uncertainty and action uncertainty as follows:

$$\begin{matrix}{U}_{n}^{R} = H ( {R}_{n} \vert Q , {Z}_{n - 1} ) \\ = {𝔼}_{{R}_{n}} [ \frac{1}{\vert {R}_{n} \vert} - log ℙ ( {R}_{n} \vert Q , {Z}_{n - 1} ) ] \\ = {𝔼}_{{R}_{n}} [ \frac{1}{\vert {R}_{n} \vert} {\sum }_{{a}_{i}}^{{R}_{n}} - log ℙ ( {a}_{i} \vert Q , {Z}_{n - 1} , {a}_{0} , ... , {a}_{i - 1} ) ]\end{matrix}$$

$$where {U}_{n}^{R}$$

is the uncertainty (either

$${U}_{n}^{T} {U}_{n}^{A} ) ,$$

R_{N} is the LLM's response, aᵢ is a token of R_{N}, H(⋅) is an entropy function, and [symbol] (⋅|⋅) is a probability.

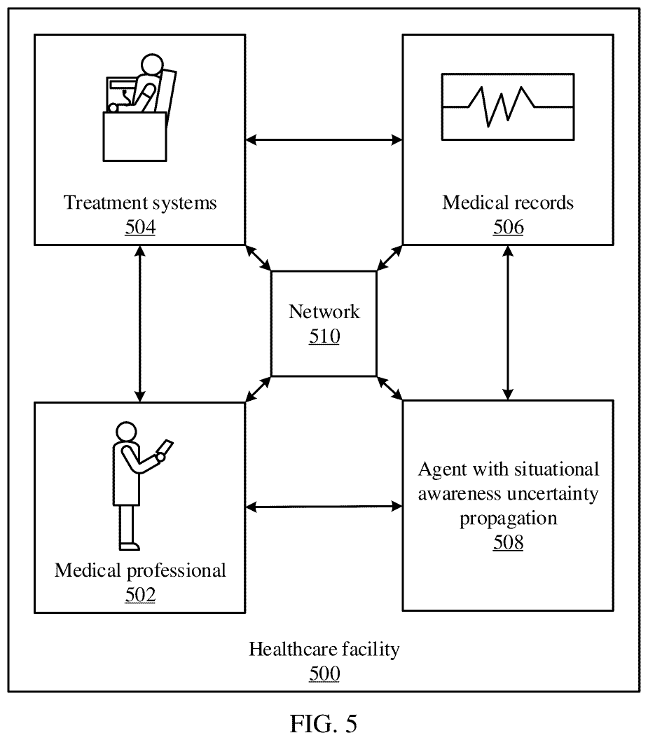

Referring now to FIG. 5, a diagram of time series analysis is shown in the context of a healthcare facility 500. An agent with situational awareness uncertainty propagation 508 may be used to aid in medical decision making. In some cases the agent 508 may be used to assess a patient's health condition, referring to the patient's medical records 506, and may make recommendations or take actions to treat the patient.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the agent with situational awareness uncertainty propagation 508, the medical professionals 502 may then make medical decisions about patient healthcare suited to the patient's needs. For example, the medical professionals 502 may make a diagnosis of the patient's health condition and may prescribe particular medications, surgeries, and/or therapies that are appropriate to the stage of a disease.

The different elements of the healthcare facility 500 may communicate with one another via a network 510, for example using any appropriate wired or wireless communications protocol and medium. Thus the agent 508 receives data from treatment systems 504, medical professionals 502, and from medical records 506, and searches the medical records 506 to output an action. The agent 508 may further coordinate with treatment systems 504 in some cases to automatically administer or alter a treatment. For example, if the agent 508 determines the patient may have a particular health condition, the agent 508 may trigger a change in treatment, such as initiating or halting the administration of a medication. The agent 508 may additionally institute an action to gather more information, such as performing tests that will reduce the uncertainty of the agent's decision. The action performed by the agent 508 may thus be guided by the estimated uncertainty.

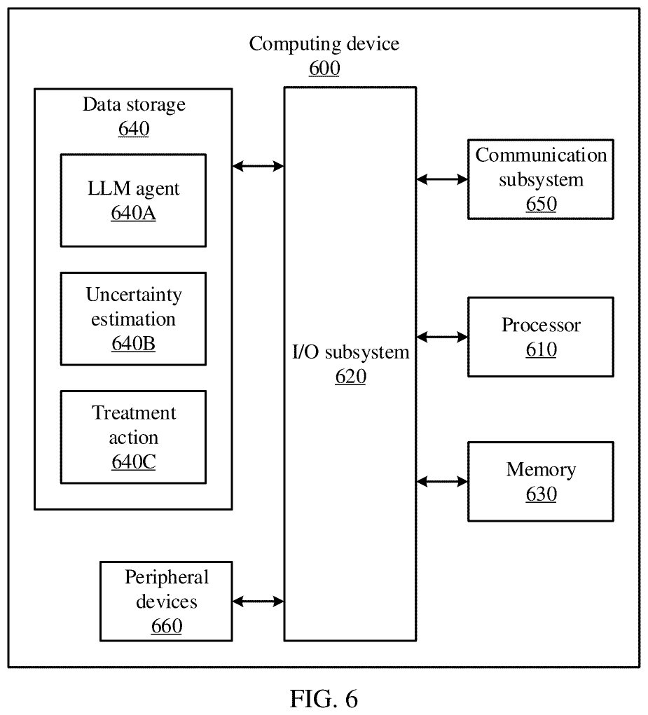

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for implementing an LLM agent, 640B for estimating uncertainty of the LLM agent's outputs, and/or 640C for performing treatment actions. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

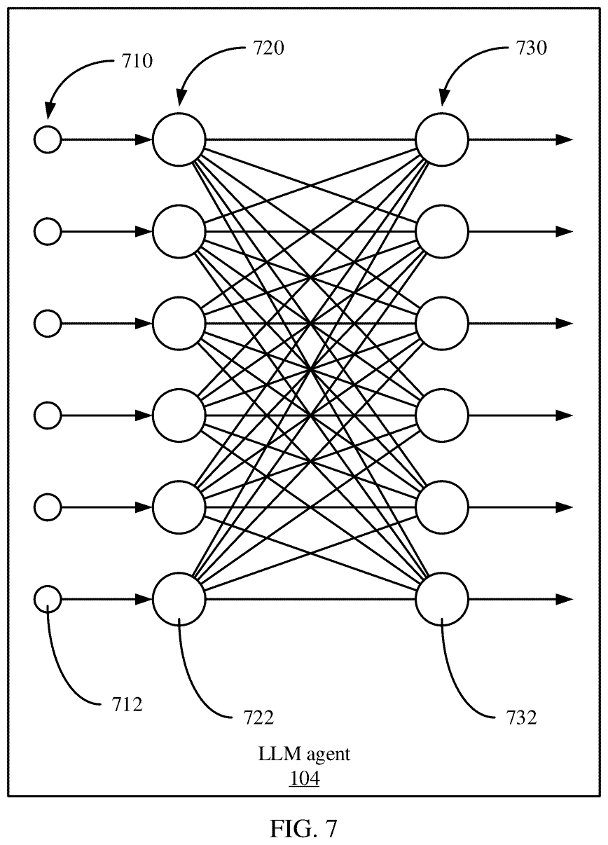

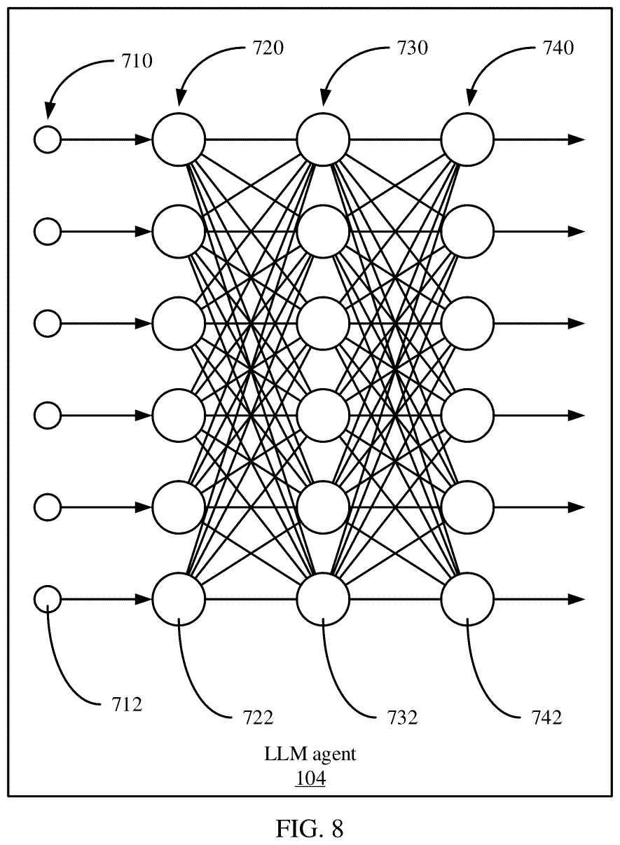

Referring now to FIGS. 7 and 8, exemplary neural network architectures are shown, which may be used to implement parts of the present machine learning models, such as the LLM agent 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
estimating situational weights for an agent based on a distance measure for a plurality of steps taken by the agent;
combining the situational weights with uncertainties from the agent for the plurality of steps to determine a total uncertainty for an action indicated by the agent; and
performing the action indicated by the agent responsive to the total uncertainty.

2. The method of claim 1, wherein the distance measure for a given step includes a first distance between an input to the agent and an agent state for the step and a second distance between a state probability matrix for the step and an observation for the step.

3. The method of claim 2, wherein estimating the situational weights includes transforming the distance measure using a hidden Markov model.

4. The method of claim 1, wherein combining the situational weights with uncertainties comprises multiplying an uncertainty for each of the plurality of steps with a respective situational weight for the respective step of the plurality of steps.

5. The method of claim 1, wherein estimating the situational weights uses a position surrogate that increases a weight value more quickly for early steps of the plurality of steps than later steps.

6. The method of claim 5, wherein estimating the situational weights combines the position surrogate with a plain distance surrogate.

7. The method of claim 1, wherein the total uncertainty is:
$${U}_{agent} = \sqrt{\frac{1}{N} {\sum }_{i = 1}^{N} ( {γ ( {W}_{i} {U}_{i} )}^{2} + ( 1 - γ ) log ( {W}_{i} {U}_{i} + 1 ) )}$$
where N is the number of the plurality of steps, γ is a weight factor, Wᵢ is the situational weight for the step i, and Uᵢ is the uncertainty from the agent for step i.

8. The method of claim 1, wherein the agent is a machine learning agent implemented by a large language model.

9. The method of claim 1, wherein the agent is prompted with an input to assist with medical decision making.

10. The method of claim 9, wherein the action indicates a treatment action for a patient that is performed responsive to a comparison of the total uncertainty to a threshold.

11. A system, comprising:
a hardware processor; and
a memory that stores a computer program which, when executed by the hardware processor, causes the hardware processor to:
estimate situational weights for an agent based on a distance measure for a plurality of steps taken by the agent;
combine the situational weights with uncertainties from the agent for the plurality of steps to determine a total uncertainty for an action indicated by the agent; and
perform the action indicated by the agent responsive to the total uncertainty.

12. The system of claim 11, wherein the distance measure for a given step includes a first distance between an input to the agent and an agent state for the step and a second distance between a state probability matrix for the step and an observation for the step.

13. The system of claim 12, wherein estimation of the situational weights includes transforming the distance measure using a hidden Markov model.

14. The system of claim 11, wherein combination of the situational weights with uncertainties comprises multiplying an uncertainty for each of the plurality of steps with a respective situational weight for the respective step of the plurality of steps.

15. The system of claim 11, wherein estimation of the situational weights uses a position surrogate that increases a weight value more quickly for early steps of the plurality of steps than later steps.

16. The system of claim 15, wherein estimation of the situational weights combines the position surrogate with a plain distance surrogate.

17. The system of claim 11, wherein the total uncertainty is:
$${U}_{agent} = \sqrt{\frac{1}{N} {\sum }_{i = 1}^{N} ( {γ ( {W}_{i} {U}_{i} )}^{2} + ( 1 - γ ) log ( {W}_{i} {U}_{i} + 1 ) )}$$
where N is the number of the plurality of steps, γ is a weight factor, Wᵢ is the situational weight for the step i, and Uᵢ is the uncertainty from the agent for step i.

18. The system of claim 11, wherein the agent is a machine learning agent implemented by a large language model.

19. The system of claim 11, wherein the agent is prompted with an input to assist with medical decision making.

20. The system of claim 19, wherein the action indicates a treatment action for a patient that is performed responsive to a comparison of the total uncertainty to a threshold.
